(19) United States
(12) Patent Application Publication
(10) Pub. No.: US 2023/0134763 A1
(43) Pub. Date: May 4, 2023

(54) CELL CULTURE METHOD AND CELL CULTURE MEMBRANE

(71) Applicant: TOYODA GOSEI CO., LTD., Kiyosu-shi (JP)

(72) Inventors: Masateru YAMAZAKI, Kiyosu-shi (JP); Sayaka HINO, Nagoya-shi (JP); Yasuhiko SHINODA, Kiyosu-shi (JP)

(21) Appl. No.: 17/975,037

(22) Filed: Oct. 27, 2022

(30) Foreign Application Priority Data
Nov. 2, 2021 (JP) 2021-179401

(51) Int. Cl. C12M 1/12; C12N 5/071

(52) U.S. Cl. CPC C12M 25/04; C12N 5/0679; C12N 5/069; C12N 2533/30

(57) ABSTRACT

A cell culture method includes: a preparation step of preparing a cell culture membrane that includes (i) a membrane body formed of a thermosetting resin and including a first surface and a second surface opposite the first surface and (ii) a plurality of through pores formed in the membrane body and penetrating from the first surface to the second surface; and a culture step of seeding and culturing cells on each of the first surface and the second surface of the cell culture membrane prepared, in the through pores, a first average pore diameter in the first surface is smaller than a second average pore diameter in the second surface and the pore density of the through pores is equal to or less than 2.0×10⁵ pores/cm².

### CROSS REFERENCE TO RELATED APPLICATIONS

This application claims priority based on Japanese Patent Application No. 2021-179401 filed on Nov. 2, 2021, the entire disclosure of which is hereby incorporated by reference.

### BACKGROUND

### Field

The present disclosure relates to cell culture methods and cell culture membranes.

### Related Art

Conventionally, in order to three-dimensionally culture adherent cells, a technique using a cell culture membrane is provided (for example, Japanese Unexamined Patent Application Publication No. 2017-29092). The culturing of cells on both surfaces of the cell culture membrane allows three-dimensional cell culture.

In order to create a state of cells closer to a state in vivo, a cell culture method and a cell culture membrane are required to be further improved.

### SUMMARY

The present disclosure is able to be realized as an aspect below.

According to one aspect of the present disclosure, a cell culture method is provided. The cell culture method includes: a preparation step of preparing a cell culture membrane that includes (i) a membrane body formed of a thermosetting resin and including a first surface and a second surface opposite the first surface and (ii) a plurality of through pores formed in the membrane body and penetrating from the first surface to the second surface; and a culture step of seeding and culturing cells on each of the first surface and the second surface of the cell culture membrane prepared, in the through pores, a first average pore diameter in the first surface is smaller than a second average pore diameter in the second surface and the pore density of the through pores is equal to or less than 2.0×10⁵ pores/cm².

According to other aspect of the present disclosure, a cell culture membrane is provided. The cell culture membrane includes: a membrane body that is formed of a thermosetting resin and includes a first surface and a second surface opposite the first surface; and a plurality of through pores that are formed in the membrane body and penetrate from the first surface to the second surface, in the through pores, a first average pore diameter in the first surface is smaller than a second average pore diameter in the second surface, the pore density of the through pores is equal to or less than 2.0×10⁵ pores/cm², the first surface is used to seed and culture a first type of cells and the second surface is used to seed and culture a second type of cells that are different from the first type of cells. In the embodiment described above, while the invasion of the cells is suppressed, the cells cultured on the second surface enter the through pores to be able to approach the cells seeded on the first surface. Hence, when the cell culture membrane is used, and thus the cells of different types are respectively cultured on the first surface and the second surface, it is possible to create a state closer to the state in vivo where heterogeneous cells interact with each other.

### BRIEF DESCRIPTION OF THE DRAWINGS

FIG. 1 is a flowchart showing steps in a cell culture method;

FIG. 2 is a perspective view of an insert to which a cell culture membrane is attached;

FIG. 3 is a cross-sectional view of the cell culture membrane;

FIG. 4 is a diagram illustrating steps in a cell culture step;

FIG. 5 is a diagram illustrating the fitting of a first jig;

FIG. 6 is a diagram illustrating the fitting of a second jig;

FIG. 7 is a diagram showing a relationship between a pore density and whether or not invasion of cells occurs;

FIG. 8 is a diagram showing the results of transepithelial electrical resistance;

FIG. 9 is a diagram showing the results of a lucifer yellow permeable assay;

FIG. 10 is a diagram illustrating a method for a transport assay; and

FIG. 11 is a diagram showing the results of a digoxin transport assay.

### DETAILED DESCRIPTION

### A. Embodiment

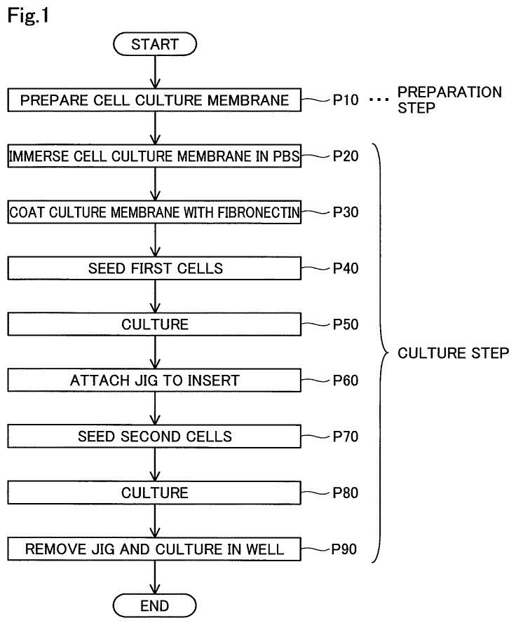

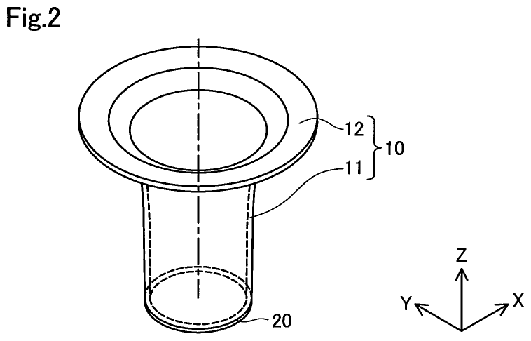

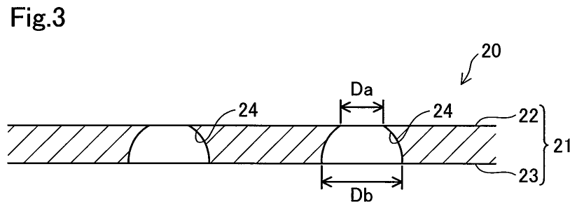

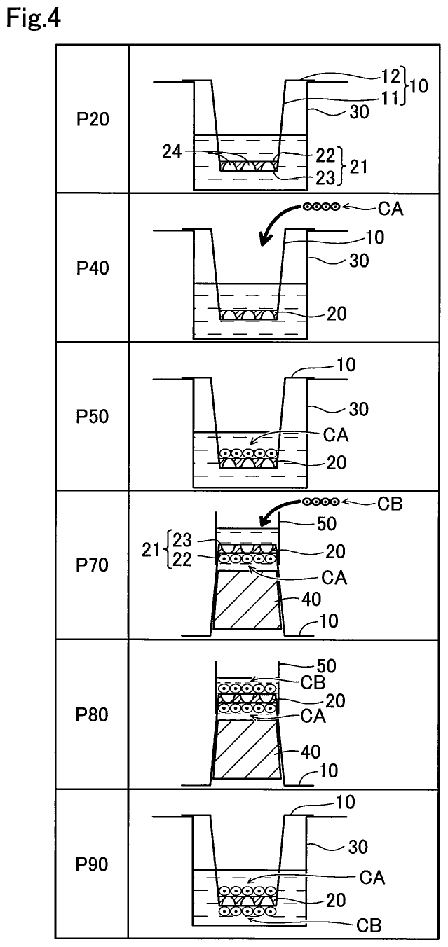

FIG. 1 is a flowchart showing steps in a cell culture method. FIG. 2 is a perspective view of an insert 10 to which a cell culture membrane 20 is attached. FIG. 3 is a cross-sectional view of the cell culture membrane 20. FIG. 4 is a diagram illustrating steps in a cell culture step. In the present embodiment, on both surfaces of the cell culture membrane 20 which is a porous membrane, different types of cells are cultured. Cells which are first seeded and cultured are referred to as first cells CA, and cells which are subsequently seeded and cultured are referred to as second cells CB. In the present embodiment, the first cells CA which serve as a first type of cells are Caco-2 (cell line derived from human colon cancer), and the second cells CB which serve as a second type of cells are human vascular endothelial cells.

As shown in FIG. 1, the cell culture step includes a preparation step P10 and a culture step from a step P20 to a step P90. In the preparation step P10, the cell culture membrane 20 is prepared. As shown in FIG. 2, in the present embodiment, the cell culture membrane 20 is used in a state where the cell culture membrane 20 is attached to the insert 10. In FIG. 2, an X-axis, a Y-axis and a Z-axis are shown which are perpendicular to each other. A direction in which the arrow of the Z-axis points is an upward direction, and the opposite direction is a downward direction. The same is true for drawings and descriptions to be given below. The insert 10 is a hanging-type insert, and is selected as necessary from TninCert made by Greiner Company, Transwell made by Corning Inc. and the like. The insert 10 includes a cylindrical portion 11 and a flange portion 12. The diameter of the cylindrical portion 11 is 6.5 mm. Specifically, the diameter of the cylindrical portion 11 is the inner diameter of the other end portion of the cylindrical portion 11 which will be described later. The flange portion 12 is in the shape of a disc, and extends from one end portion of the cylindrical portion 11 in an outer diameter direction. The circular cell culture membrane 20 is attached to the other end portion of the cylindrical portion 11 so as to cover the end portion. In the present embodiment, the cell culture membrane 20 is attached such that a first surface 22 to be described later is directed to the inside of the cylindrical portion 11.

The cell culture membrane 20 is formed of a thermosetting resin. Specifically, polyurethane, polyurea, a silicone resin, a phenolic resin, an epoxy resin, unsaturated polyester, polyimide or the like may be used. In the present embodiment, polyurethane is used. The membrane thickness of the cell culture membrane 20 is equal to or greater than 3 μm and equal to or less than 7 μm.

As shown in FIG. 3, the cell culture membrane 20 includes: a membrane body 21 which includes the first surface 22 and a second surface 23 opposite the first surface 22; and a plurality of through pores 24 which are formed in the membrane body 21. The through pores 24 are pores which penetrate from the first surface 22 to the second surface 23. The pore diameter of the through pore 24 in the first surface 22 is different from that of the through pore 24 in the second surface 23. The pore diameter of the through pore 24 in the first surface 22 is referred to as a first pore diameter Da, and the pore diameter of the through pore 24 in the second surface 23 is referred to as a second pore diameter db. The first pore diameter Da is smaller than the second pore diameter db. A first average pore diameter which is the average value of the first pore diameters Da of the through pores 24 is different from a second average pore diameter which is the average value of the second pore diameters db in the second surface 23. The first average pore diameter is smaller than the second average pore diameter. The first average pore diameter is preferably equal to or less than 7 μm. In this way, it is possible to suppress, after the first cells CA are seeded on the cell culture membrane 20, the movement of the first cells CA passing through the through pores 24 to a surface on a side opposite to a surface to which the first cells CA are seeded. In the following description, the movement of the first cells CA passing through the through pores 24 to the surface on the side opposite to the surface to which the first cells CA are seeded may be referred to as “cell invasion”. The first average pore diameter is further preferably equal to or greater than 3 μm and equal to or less than 5 μm. In this way, when the size of the first cell CA at the time of seeding is about 5 μm, the first average pore diameter is set equal to or less than 5 μm, and thus the invasion of the first cells CA is suppressed whereas the first average pore diameter is set equal to or greater than 3 μm, and thus it is possible to easily bring the first cells CA into contact with the second cells CB. In other words, a case where the first average pore diameter is equal to or greater than 3 μm refers to a case where the second average pore diameter is greater than 3 μm. In this case, the second cells CB easily enter the through pores 24 from the side of the second surface 23, and thus it is possible to easily bring the first cells CA into contact with the second cells CB.

Here, the first average pore diameter is a value which is determined by applying light to the first surface 22 directed upward in a state where the first surface 22 is directed upward and the second surface 23 is directed downward and performing observation with a microscope. Since the light is not reflected off the through pores 24, the through pores 24 are visually recognized in black. Specifically, the circle-equivalent diameters of all the through pores 24 which are observed in a specific field of view are measured, and the average value of the measured values is the first average pore diameter. The circle-equivalent diameter refers to the diameter of a perfect circle which corresponds to the area of the through pore 24. The second average pore diameter is a value which is determined in the same manner. Specifically, the second average pore diameter is a value which is determined by applying light to the second surface 23 directed upward in a state where the second surface 23 is directed upward and the first surface 22 is directed downward and performing observation with the microscope.

The pore density of the through pores 24 is equal to or less than 2.0×10⁵ pores/cm². In this way, it is possible to suppress the invasion of the first cells CA. The pore density of the through pores 24 is equal to or less than 1.5×10⁵ pores/cm². In this way, it is possible to further suppress the bypassing of the first cells CA. It is likely that it is difficult to fix the second cells CB on the surface of the cell culture membrane 20 on which the first cells CA are fixed. Hence, the invasion of the first cells CA is suppressed, and thus the first cells CA and the second cells CB are able to be satisfactorily cultured on both surfaces of the cell culture membrane 20. In this way, it is possible to form a state closer to a human body.

It is possible to manufacture the cell culture membrane 20 by supplying water vapor to an uncured polyurethane raw material which is formed in the shape of a thin plate and curing the uncured polyurethane raw material while foaming it. Since in the manufacturing method described above, the through pores 24 are formed by foaming, it is possible to manufacture the cell culture membrane 20 in which the first pore diameter Da is different from the second pore diameter db.

In the present embodiment, the shape of the through pore 24 in the first surface 22 is close to a perfect circle. Specifically, although when a rectangle circumscribing the through pore 24 is drawn in the observation of the microscope described above, the average value of the ratio of a long side to a short side is about 1.1, the present embodiment is not limited to this configuration. Although in the present embodiment, the second average pore diameter which is the average value of the second pore diameters db of the through pores 24 is equal to or greater than 4 μm and equal to or less than 7 μm, the present embodiment is not limited to this configuration.

In the step P20 of FIG. 1, the cell culture membrane 20 is immersed in phosphate buffered saline (PBS) and is incubated overnight. The conditions of the incubation are a temperature of 37° C. and a CO₂ concentration of 5%. Specifically, as shown in the step P20 of FIG. 4, the insert 10 is put into one well 30 in a 24-well plate. Since the diameter of the outer circumference of the flange portion 12 is larger than the diameter of the well 30, the flange portion 12 abuts on the peripheral surface of the well 30, and thus the cell culture membrane 20 is held in a state where the cell culture membrane 20 is separated from the bottom surface of the well 30. The cell culture membrane 20 is incubated overnight in the step P20, and thus it is possible to remove air bubbles adhered to the surface of the cell culture membrane 20 when the cell culture membrane 20 is immersed in the phosphate buffered saline.

As shown in the step P30 of FIG. 1, the first surface 22 and the second surface 23 of the cell culture membrane 20 are coated with human fibronectin. In this way, it is possible to enhance the adhesion rate of the human vascular endothelial cells which are the second cells CB to be seeded second.

As shown in FIGS. 1 and 4, in a step P40 and a step P50 which serve as a first step, the first cells CA are seeded and cultured on the first surface 22. In the step P40, Caco-2 which is the first cell CA is seeded into the inside of the insert 10, that is, to the first surface 22 of the cell culture membrane 20. As shown in FIGS. 1 and 4, in the step P50, the first cells CA are cultured for three days. In the present embodiment, the number of first cells CA which are seeded is 50000. The conditions of the incubation are a temperature of 37° C. and a CO₂ concentration of 5%. As a culture medium, D-MEM is used in which no serum is provided and to which bovine serum albumin (BSA) at a concentration of 5 μM is added.

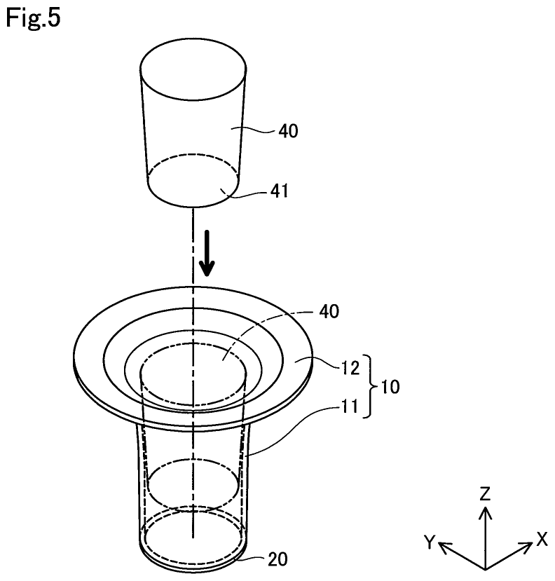

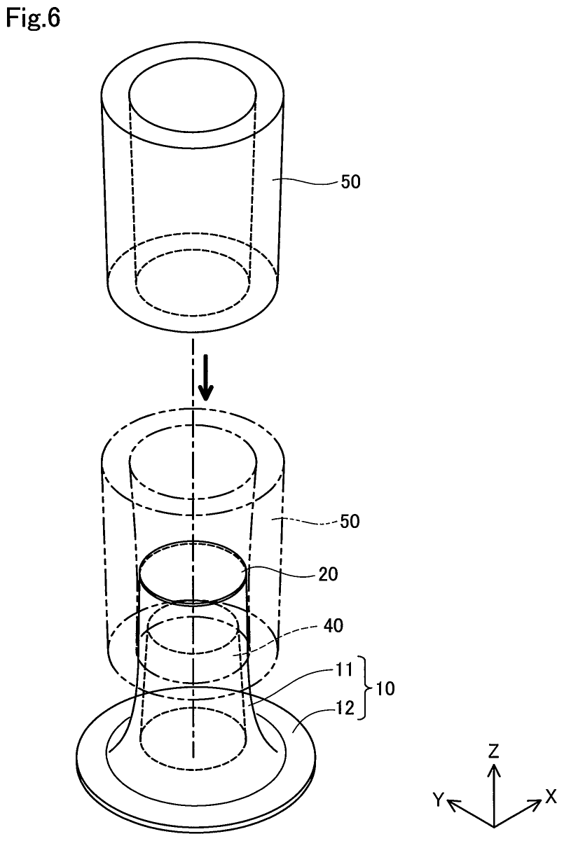

In a step P60 shown in FIG. 1, a first jig 40 and a second jig 50 are attached to the insert 10. FIG. 5 is a diagram illustrating the fitting of the first jig 40 to the insert 10. FIG. 6 is a diagram illustrating the fitting of the second jig 50 to the insert 10. Specifically, as shown in FIG. 5, the first jig 40 is first inserted into the insert 10. The first jig 40 is flexible and has a cylindrical shape in which its diameter is decreased toward a tip 41. The first jig 40 has such a size that, in the middle of inserting the first jig 40 into the cylindrical portion 11, the outer circumferential surface of the first jig 40 makes intimate contact with the inner circumferential surface of the cylindrical portion 11. Hence, immediately before the tip 41 of the first jig 40 abuts on the cell culture membrane 20, in a state where the outer circumferential surface of the first jig 40 is in intimate contact with the inner circumferential surface of the cylindrical portion 11, the first jig 40 is held by the cylindrical portion 11. Then, in a state where the culture medium is put in the insert 10, the interior of the insert 10 is sealed by the first jig 40. Then, the insert 10 to which the first jig 40 is fitted is inverted, and as shown in FIG. 6, the second jig 50 is fitted to the outer circumference of the insert 10 so as to cover the cell culture membrane 20. The second jig 50 is flexible and has a cylindrical shape. In a state where the second jig 50 is fitted, the end portion of the second jig 50 which is more distant from the flange portion 12, that is, the end portion in the upward direction is fitted so as to be separated from the cell culture membrane 20. In this way, a space for holding the culture medium is formed by the cell culture membrane 20 and the inner circumferential surface of the second jig 50.

As shown in FIGS. 1 and 4, in a step P70 and a step P80 which serve as a second step, the second cells CB are seeded and cultured on the second surface 23. In the step P70 shown in FIG. 1, the second cells CB are seeded into the inside of the second jig 50, that is, to the second surface 23 of the cell culture membrane 20. As shown in FIGS. 1 and 4, in the step P80, the first cells CA and the second cells CB are cultured for four hours. In the present embodiment, the number of second cells CB which are seeded is 100000. The same human vascular endothelial cell culture medium is used for the first cells CA and the second cells CB. As shown in FIGS. 1 and 4, in the step P90, the first jig 40 and the second jig 50 are removed from the insert 10. Thereafter, the insert 10 is put into the well 30 and is cultured for three days with the culture medium changed daily. In the culture, the culture medium which is suitable for each of the first cell CA and the second cell CB is used. By the cell culture method described above, double-layer culture of a Caco-2 cell layer and a vascular endothelial cell layer is realized. Then, by the cell culture method according to the present embodiment, it is possible to make an organ model and specifically, a small intestine model.

In the embodiment described above, the cell culture membrane 20 is prepared in the preparation step P10. The cell culture membrane 20 includes: the membrane body 21 which is formed of the thermosetting resin and includes the first surface 22 and the second surface 23; and a plurality of through pores 24 which are formed in the membrane body 21. In the through pores 24, the first average pore diameter in the first surface 22 is smaller than the second average pore diameter in the second surface 23, and the pore density is equal to or less than 2.0×10⁵ pores/cm². In the culture step, the cells are seeded and cultured on each of the first surface 22 and the second surface 23 of the cell culture membrane 20. The pore density is equal to or less than 2.0×10⁵ pores/cm², and thus it is possible to suppress the cell invasion, that is, the movement of the first cells CA seeded on the first surface 22 through the through pores 24 to the second surface 23. Furthermore, since the second average pore diameter is larger than the first average pore diameter, the second cells CB seeded on the second surface 23 enter the through pores 24 to be able to approach the first cells CA. Hence, when the first cells CA and the second cells CB of different types are respectively cultured on the first surface 22 and the second surface 23, the distance between the second cells CB and the first cells CA is reduced, and thus it is possible to create a state closer to a state in vivo where heterogeneous cells interact with each other. Therefore, it is possible to make an organ model which has a satisfactory transport capability and a satisfactory barrier property. The organ model described above is used, and thus it is possible to reduce a time for drug development, with the result that it is possible to reduce the costs of the drug development.

The culture step includes: the step P40 and the step P50 in which the first cells CA are seeded and cultured on the first surface 22; and the step P70 and the step P80 in which the second cells CB are seeded and cultured on the second surface 23. In this way, it is possible to satisfactorily perform the culture on the first surface 22 and the culture on the second surface 23.

The first cells CA are intestinal epithelial cells, and the second cells CB are vascular endothelial cells. In this way, the first cells CA and the second cells CB which are cultured are able to be used as an organ model.

### B. Other Embodiments

(B1) In the embodiment described above, the first cells CA and the second cells CB of different types are respectively cultured on the first surface 22 and the second surface 23 of the cell culture membrane 20. There is no limitation to this configuration, and the cells of the same type may be cultured on the first surface 22 and the second surface 23, two or more types of cells may be cultured on each of the first surface 22 and the second surface 23. The first cells CA may be cultured on the second surface 23, and the second cells CB may be cultured on the first surface 22. The first cells CA, that is, the cells which are first seeded and cultured may be human vascular endothelial cells, and the second cells CB which are subsequently seeded and cultured may be Caco-2.

(B2) In the embodiment described above, the first cells CA are Caco-2, and the second cells CB are vascular endothelial cells. The first cells CA are not limited to Caco-2, and may be other intestinal epithelial cells. Examples of the other intestinal epithelial cells which may be used include HT-29 cells, primary small intestinal epithelial cells, iPS-derived small intestinal epithelial cells, stem cells, Paneth cells, crypt cells, mucus-secreting cells and the like. In the embodiment described above, in order to produce a small intestine model, human vascular endothelial cells are used as the second cells CB. The second cells CB are preferably selected according to the organ model which is produced. Examples of the other organ model include a cerebrovascular model, a liver model, a kidney model, a lung model and the like.

The present disclosure is not limited to the embodiments described above, and may be realized in various configurations without departing from the spirit thereof. For example, the technical features of any of the above embodiments and their modifications may be replaced or combined appropriately, in order to solve part or all of the problems described above or in order to achieve part or all of the advantageous effects described above. When the technical features are not described as essential features in the present specification, they may be deleted as necessary. For example, the present disclosure may be realized in embodiments below.

(1) According to one embodiment of the present disclosure, a cell culture method is provided. The cell culture method includes: a preparation step of preparing a cell culture membrane that includes (i) a membrane body formed of a thermosetting resin and including a first surface and a second surface opposite the first surface and (ii) a plurality of through pores formed in the membrane body and penetrating from the first surface to the second surface; and a culture step of seeding and culturing cells on each of the first surface and the second surface of the cell culture membrane prepared, in the through pores, a first average pore diameter in the first surface is smaller than a second average pore diameter in the second surface and the pore density of the through pores is equal to or less than 2.0×10⁵ pores/cm². In the embodiment described above, while the invasion of the cells is suppressed, the cells seeded on the second surface are able to make contact with the cells cultured on the first surface through the through pores. Here, the cell invasion refers to the movement of the cells seeded on one of the first surface and the second surface through the through pores to the other surface. Hence, when the cells of different types are respectively cultured on the first surface and the second surface, it is possible to create a state closer to a state in vivo where heterogeneous cells interact with each other. In this way, it is possible to make an organ model which has a satisfactory transport capability and a satisfactory barrier property.

(2) In the cell culture method of the embodiment described above, in the culture step, the type of the cells seeded and cultured on the first surface may be different from the type of the cells seeded and cultured on the second surface. In the embodiment described above, the cells of different types are respectively cultured on the first surface and the second surface, and thus it is possible to create a state closer to the state in vivo where heterogeneous cells interact with each other.

(3) In the cell culture method of the embodiment described above, the culture step may include: a first step of seeding and culturing the cells on the first surface; and a second step of seeding and culturing, after the first step, the cells on the second surface. In the embodiment described above, it is possible to satisfactorily perform the culture on the first surface and the culture on the second surface.

(4) In the cell culture method of the embodiment described above, first cells seeded and cultured on the first surface may be intestinal epithelial cells, and second cells seeded and cultured on the second surface may be vascular endothelial cells. In the embodiment described above, it is possible to make an organ model.

(5) In the cell culture method of the embodiment described above, the first average pore diameter may be equal to or less than 7 μm. In the embodiment described above, when the cells having a size of about 10 μm are cultured, it is possible to further suppress the invasion of the cells.

(6) In the cell culture method of the embodiment described above, the first average pore diameter may be equal to or greater than 3 μm and equal to or less than 5 μm. In the embodiment described above, when the cells having a size of about 5 μm at the time of seeding are seeded, the size is equal to or less than 5 μm, and thus the invasion of the cells is able to be further suppressed whereas the size is equal to or greater than 3 μm, and thus it is possible to easily bring the cells seeded on the second surface into contact with the cells cultured on the first surface.

(7) In the cell culture method of the embodiment described above, the pore density may be equal to or less than 1.5×10⁵ pores/cm². In the embodiment described above, the bypassing of the cells is able to be further suppressed.

(8) According to other embodiment of the present disclosure, a cell culture membrane is provided. The cell culture membrane includes: a membrane body that is formed of a thermosetting resin and includes a first surface and a second surface opposite the first surface; and a plurality of through pores that are formed in the membrane body and penetrate from the first surface to the second surface, in the through pores, a first average pore diameter in the first surface is smaller than a second average pore diameter in the second surface, the pore density of the through pores is equal to or less than 2.0×10⁵ pores/cm², the first surface is used to seed and culture a first type of cells and the second surface is used to seed and culture a second type of cells that are different from the first type of cells. In the embodiment described above, while the invasion of the cells is suppressed, the cells cultured on the second surface enter the through pores to be able to approach the cells seeded on the first surface. Hence, when the cell culture membrane is used, and thus the cells of different types are respectively cultured on the first surface and the second surface, it is possible to create a state closer to the state in vivo where heterogeneous cells interact with each other.

### C. Examples and Comparative Examples

### C1. Evaluation of Bypassing

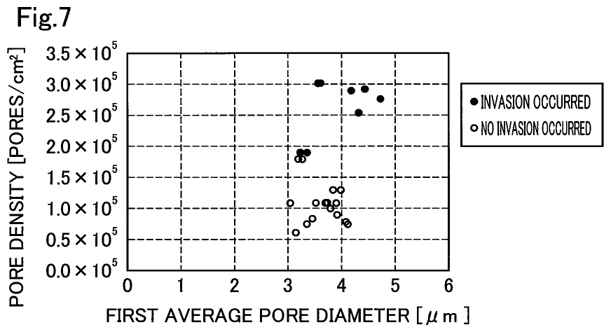

FIG. 7 is a diagram showing a relationship between the pore density and whether or not the invasion of cells occurs. Cell culture membranes which had various first average pore diameters and pore densities and were formed of polyurethane were prepared. The membrane thicknesses were equal to or greater than 3 μm and equal to or less than 7 μm. The second average pore diameters were equal to or greater than 4 μm and equal to or less than 7 μm. The prepared cell culture membranes were used, and thus the same step as the cell culture step of the embodiment described above was performed, with the result that the cells were cultured. On the first surface 22, Caco-2 was cultured, and on the second surface 23, human vascular endothelial cells were cultured. Thereafter, Caco-2 and the human vascular endothelial cells were stained, and whether or not the bypassing of Caco-2 to the second surface 23 occurred was evaluated by fluorescence inverted microscopy. The size of Caco-2 at the time of seeding was about the same as that of a cell nucleus, that is, about 5 μm. The size of Caco-2 at the time of fixing was about 10 μm. Hence, the cell culture membranes in which the first average pore diameter was equal to or less than 5 μm were used, and thus it was possible to suppress the bypassing of Caco-2 from the first surface 22 to the second surface 23 as a result of Caco-2 being seeded and passed through the through pores 24. The cell culture membranes in which the first average pore diameter was equal to or greater than 3 μm were used, and thus it was possible to easily bring Caco-2 into contact with the vascular endothelial cells. Therefore, the inventors conducted evaluations only on the cell culture membranes in which the first average pore diameter was equal to or greater than 3 μm and equal to or less than 5 μm.

As shown in FIG. 7, it was found that when the cell culture membranes were used in which the first average pore diameter was equal to or greater than 3 μm and equal to or less than 5 μm and the pore density was equal to or less than 2.0×10⁵ pores/cm², the invasion of Caco-2 was suppressed. Furthermore, it was found that when the cell culture membranes were used in which the pore density was equal to or less than 1.5×10⁵ pores/cm², the invasion of Caco-2 was further suppressed.

### C2. Evaluation of Barrier Property

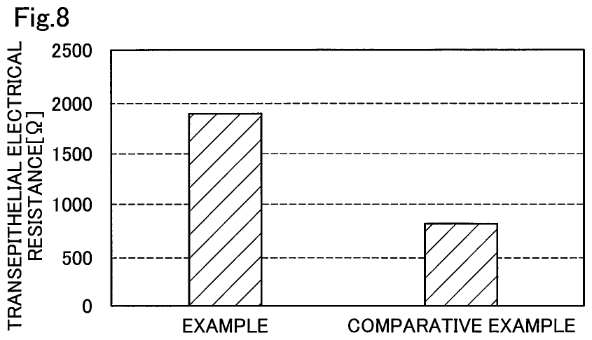

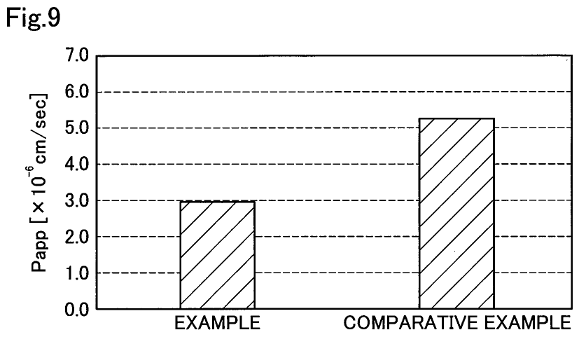

FIG. 8 is a diagram showing the results of transepithelial electrical resistance. FIG. 9 is a diagram showing the results of a lucifer yellow permeable assay. In Example, results were shown using the cell culture membrane in which the first average pore diameter was equal to or greater than 3 μm and equal to or less than 5 μm, the pore density was equal to or less than 2.0×10⁵ pores/cm² and cell invasion after the cell culture described above was performed was not confirmed. The number of samples was two, and in FIGS. 8 and 9, the average values were shown. In Comparative Example, results were shown using the cell culture membrane which was formed of PET (Polyethylene terephthalate) and in which the pore diameter was 3 μm, the pore density was 2.0×10⁶ pores/cm² and the membrane thickness was 10 μm. In Comparative Example, since the through pores were formed by laser, the pore diameter in the first surface of the cell culture membrane was about the same as that in the second surface. In the lucifer yellow permeable assay, lucifer yellow was added to the inside of the insert 10, and after being incubated, a solution between the well 30 and the insert 10 was collected and measured, with the result that a permeation coefficient was calculated.

As shown in FIG. 8, the transepithelial electrical resistance of Example was higher than the transepithelial electrical resistance of Comparative Example. As shown in FIG. 9, the permeation coefficient of the lucifer yellow in Example was less than that of the lucifer yellow in Comparative Example. Hence, it was found that the barrier property of Example was higher than that of Comparative Example. It is considered that this is because in Example, Caco-2 and the vascular endothelial cells were satisfactorily cultured, in the cell culture membrane of Example, Caco-2 made contact with the vascular endothelial cells to create a state closer to the state in vivo than in Comparative Example. By contrast, it is considered that since in the cell culture membrane of Comparative Example, the pore diameter in the first surface was about the same as that in the second surface, the vascular endothelial cells were prevented from entering the through pores, and thus there was no or few opportunities that Caco-2 made contact with the human vascular endothelial cells.

### C3. Evaluation of Selective Transport Ability

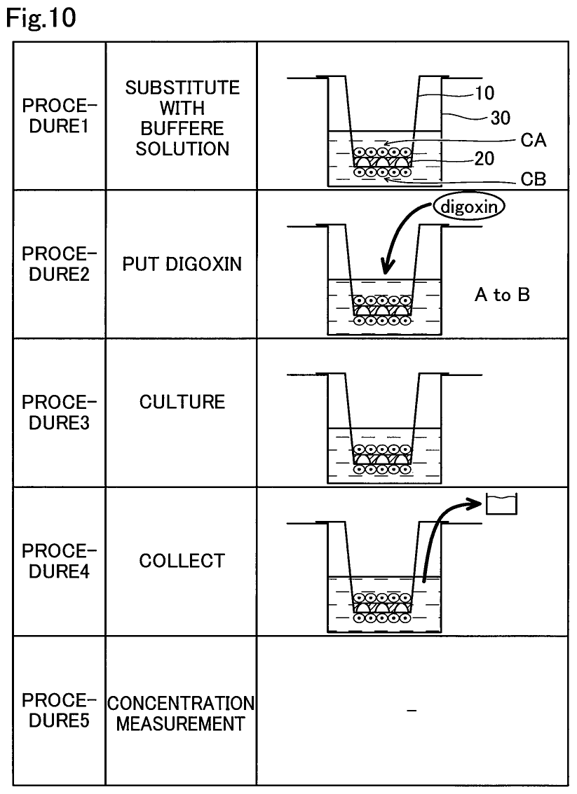

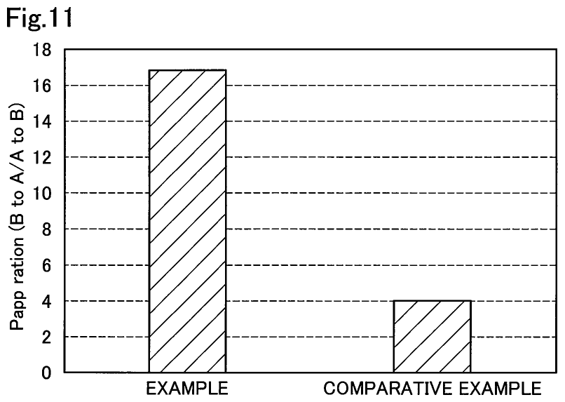

FIG. 10 is a diagram illustrating a method for a transport assay. FIG. 11 is a diagram showing the results of a digoxin transport assay. In Example and Comparative Example shown in FIG. 11, the same samples as those for the evaluation of the barrier property in Example and Comparative Example were used. Specifically, FIG. 10 is a diagram showing a procedure for a transport assay (A to B) from a Caco-2 cell side (apical side) to a human vascular endothelial cell membrane side (basal side). In procedure 1 of FIG. 10, a culture medium was substituted with a Hanks balanced salt solution (HBSS) and was incubated for 20 minutes. The conditions of the incubation were a temperature of 37° C. and a CO₂ concentration of 5%. In procedure 2, a digoxin solution was added into the insert 10. The concentration of the digoxin was adjusted to be 5 μM. In procedure 3, the solution was cultured for 2 hours. The conditions of the incubation were a temperature of 37° C. and a CO₂ concentration of 5%. In procedure 4, 100 μL of the solution was collected twice from between the well 30 and the insert 10. In procedure 5, the digoxin concentration was measured with a liquid chromatograph mass spectrometer (LC/MS/MS), and thus a transport speed was calculated. A transport test (B to A) from the vascular endothelial cell side to the Caco-2 side was performed in the same manner. Specifically, in procedure 2, the digoxin solution was added between the well 30 and the insert 10, in procedure 4, the digoxin concentration of the solution collected from the well 30 was measured and a transport speed was calculated. A ratio between the transport speeds was calculated by dividing the transport speed of B to A by the transport speed of A to B.

As shown in FIG. 11, it was found that in Example, the ratio between the transport speeds was higher than in Comparative Example and the selective transport ability of the digoxin was higher than that in Comparative Example. It is considered that this is because as in the results of the barrier property described above, as compared with the cell culture membrane used in Comparative Example, in the cell culture membrane used in Example, Caco-2 made contact with the human vascular endothelial cells to create a state closer to the state in vivo.

What is claimed is:

1. A cell culture method comprising:
a preparation step of preparing a cell culture membrane that includes (i) a membrane body formed of a thermosetting resin and including a first surface and a second surface opposite the first surface and (ii) a plurality of through pores formed in the membrane body and penetrating from the first surface to the second surface; and
a culture step of seeding and culturing cells on each of the first surface and the second surface of the cell culture membrane prepared,
wherein in the through pores, a first average pore diameter in the first surface is smaller than a second average pore diameter in the second surface, and
a pore density of the through pores is equal to or less than 2.0×10⁵ pores/cm².

2. The cell culture method according to claim 1,
wherein in the culture step, a type of the cells seeded and cultured on the first surface is different from a type of the cells seeded and cultured on the second surface.

3. The cell culture method according to claim 1,
wherein the culture step includes:
a first step of seeding and culturing the cells on the first surface; and
a second step of seeding and culturing, after the first step, the cells on the second surface.

4. The cell culture method according to claim 2,
wherein first cells seeded and cultured on the first surface are intestinal epithelial cells, and
second cells seeded and cultured on the second surface are vascular endothelial cells.

5. The cell culture method according to claim 1,
wherein the first average pore diameter is equal to or less than 7 μm.

6. The cell culture method according to claim 5,
wherein the first average pore diameter is equal to or greater than 3 μm and equal to or less than 5 μm.

7. The cell culture method according to claim 1,
wherein the pore density is equal to or less than 1.5×10⁵ pores/cm².

8. A cell culture membrane comprising:
a membrane body that is formed of a thermosetting resin and includes a first surface and a second surface opposite the first surface; and
a plurality of through pores that are formed in the membrane body and penetrate from the first surface to the second surface,
wherein in the through pores, a first average pore diameter in the first surface is smaller than a second average pore diameter in the second surface,
a pore density of the through pores is equal to or less than 2.0×10⁵ pores/cm²,
the first surface is used to seed and culture a first type of cells and
the second surface is used to seed and culture a second type of cells that are different from the first type of cells.
